(19) United States
(12) Patent Application Publication
(10) Pub. No.: US 2025/0104862 A1
(43) Pub. Date: Mar. 27, 2025

(54) AN ADAPTIVE DISEASE PREDICTION SYSTEM IN AN AI SYMPTOM-CHECKER

(71) Applicant: JIO PLATFORMS LIMITED, Ahmedabad (IN)

(72) Inventors: Chetan B. BHATT, Mumbai (IN); Shailesh KUMAR, Hyderabad (IN); Amrita Virendra VERMA, Hyderabad (IN); Kalyan TADEPALLI, Mumbai (IN); Ritesh SHAH, Hyderabad (IN); Vamshi CHITTALA, Hyderabad (IN); Hareesh KUMAR, Hosur (IN); Neeraj YOGENDRA, Hyderabad (IN)

(73) Assignee: JIO PLATFORMS LIMITED, Ahmedabad (IN)

(21) Appl. No.: 18/728,869

(22) PCT Filed: Nov. 25, 2022
(86) PCT No.: PCT/IB2022/061426

§ 371 Date: Jul. 13, 2024

(30) Foreign Application Priority Data
Nov. 29, 2021 (IN) 202121055066

(51) Int. Cl. G16H 50/20; G16H 10/20; G16H 50/70

(52) U.S. Cl. CPC G16H 50/20; G16H 10/20; G16H 50/70

(57) ABSTRACT

The present invention provides solution to the above-mentioned problem in the art by providing a system and a method for an efficient and adaptive disease prediction system as a part of an expert-based online AI symptom-checker. The system may diagnose a wide variety of diseases accurately in an under 3-minute chatbot based conversation. The system architecture comprises of state-based components namely dialogue, patient, utterance, disease and symptom state. The disease prediction module may use a complex knowledge graph coupled with the state information to predict disease scores, predict disease scores based on various factors such as symptom coverage, relative disease importance, diseases priors, risk factors, lab tests, body organs and body systems and further provide mechanisms to arrive at a convergence based on factors such as confidence scores, questions asked and the like.

### FIELD OF INVENTION

The embodiments of the present disclosure generally relate to the field of disease prediction. More particularly, the present disclosure relates generally to system and method providing a built state-based design of an artificial intelligence (AI) engine for harnessing a knowledge graph for providing health diagnostics as a service.

### BACKGROUND OF THE INVENTION

The following description of related art is intended to provide background information pertaining to the field of the disclosure. This section may include certain aspects of the art that may be related to various features of the present disclosure. However, it should be appreciated that this section be used only to enhance the understanding of the reader with respect to the present disclosure, and not as admissions of prior art.

As the population ages, health and fitness become important. With the advent of Internet, access to information, including health-related information, continues to increase exponentially. Individuals currently have instant online access, from a wide variety of sources, to personal and aggregate information relating to patient histories, test results, physical and mental fitness, nutrition, health supplements, and a vast array of health research, as well as methods and devices for measuring, diagnosing and treating various conditions or simply enhancing overall wellness. There are online symptom checkers that help users diagnose the most likely disease(s) based on the symptoms provided by them. In the healthcare domain, a symptom-based triage is known to be a critical and complex use-case due to various factors such as the impact and subjectivity of diagnoses, knowledge engineering of the vast medical information and the inferencing required thereof. Online symptom checkers find a significant application in providing quick diagnoses to patients who do not have easy access to health facilities. Such tools can also be referred to by medical students to learn differential diagnosis. The existing online symptom-checkers or diagnostics-as-a-service products make use of medical ontologies and web-data coupled with knowledge-based or empirical based approaches to achieve accurate disease predictions. They are available either as products or online web applications. Few employ an interactive methodology in the form of question-answers in a chatbot-like setting whereas some predict diseases based only on initial symptoms entered by the user. The key factors that interactive symptom-checkers are typically evaluated upon are as follows.
Accuracy of the differential diagnosis
Coverage of diseases and symptoms
Correctness/Relevance of questions asked
Order in which the questions are asked
Number of questions that need to be answered

Existing online symptom checkers employ various automatic methods to elicit the required medical data from the web. Often due to inconsistent or limited structured datasets on the web, this potentially leads to an inaccurate knowledge base at the outset. Furthermore, such data when used to train various machine learning models tend to propagate these errors eventually resulting in less accurate diagnosis.

Patent US2019355082A1 provides symptoms and methods for predicting the incidence of a disease or disorder are disclosed. A system for predicting the incidence of a disease or disorder includes a web-based symptom checker for producing a structured dataset, a data analysis component for producing a multivariate dataset from the structured dataset, and a feature construction component for producing a linear combination of orthogonal symbols representative of a disease or disorder. A method for predicting the incidence of a disease or disorder includes producing a multivariate dataset representing patient symptom counts, performing feature construction analysis on the multivariate dataset, creating a time series model using weekly illness incidence data, and applying the time series model to new illness incidence data to predict the incidence of a disease or disorder in the future. But the system generates a structured dataset by on-line symptom checking diagnostic like WebMD's Symptom Checker and further transformed into a multivariate dataset, which can affect precision or accuracy of the base data being used to train various models. Another approach creates a time-series model using weekly illness incidence data from the web, which can suffer precision or accuracy of the base data being used to train various models.

There is therefore a need in the art to provide for a state-based design of the proposed AI-engine and the expert curated medical knowledge to alleviate issues in prior art and facilitate prediction of a wide variety of diseases accurately.

### Objects of the Present Disclosure

Some of the objects of the present disclosure, which at least one embodiment herein satisfies are as listed herein below.

It is an object of the present disclosure to provide a system and a method that facilitates retrieval of patient's personal and demographic information such as age and gender for better filtering on possible diagnosis.

It is an object of the present disclosure to provide a system and a method that enables effective usage of the schema design comprising of primary entities in the form of diseases and symptoms (further qualified into attributes and values).

It is an object of the present disclosure to provide a working-memory Knowledge graph to represent the interconnections between the primary entity nodes and secondary entity nodes in the form of curated weighted edges.

It is an object of the present disclosure to provide a working-memory knowledge graph design allows for modelling dependencies between symptom attributes and symptom-attribute values further providing a possibility to ask conditional questions.

It is an object of the present disclosure to provide a Patient State monitoring module which contains complete information that the patient has shared with us, including but not limited to age, gender, dominating/bothering symptoms, responses to the questions asked.

It is an object of the present disclosure to provide a scoring mechanism for differential diagnosis from the above-mentioned Patient State.

It is an object of the present disclosure to provide a diagnostics-as-a-service that render immediate online diagnostic solutions to the people, even to the remotest areas of the Indian populace.

It is an object of the present disclosure to provide a diagnostics-as-a-service that assist medical students to learn differential diagnosis.

It is an object of the present disclosure to provide a diagnostics-as-a-service that presenting itself as a flagship product.

It is an object of the present disclosure to provide a system that benefits healthcare by collectively increasing efficiency, providing quick diagnostics to less privileged patients, and reduce overall costs.

It is an object of the present disclosure to provide a system that serves as a reference to medical students assisting them in carrying out a triage with the patient and diagnose accurately.

### SUMMARY

This section is provided to introduce certain objects and aspects of the present disclosure in a simplified form that are further described below in the detailed description. This summary is not intended to identify the key features or the scope of the claimed subject matter.

In an aspect, the present disclosure provides for a system facilitating a response of prediction of a disease. The system may include one or more processors operatively coupled to a plurality of first computing devices, the one or more processors coupled with a memory that stores instructions which when executed by the one or more processors causes the system to receive a set of input queries from one or more users associated with the plurality of first computing devices, the set of input queries pertaining to one or more symptoms associated with a disease. The system may be further configured to extract a first set of attributes from the received set of input queries, the first set of attributes pertaining to the one or more symptoms of the disease and then extract a second set of attributes from the received set of input queries, the second set of attributes pertaining to demographic conditions and location. Based on the extracted first and second attributes, the system may be further configured to generate, by using an artificial intelligence (AI) engine, a trained model configured to process each input query and based on the trained model, the system may be configured to map, by using the AI engine, each input query with a respective response that may be associated with an information service that may be further associated with a knowledge graph comprising interconnections between a target disease and the one or more symptoms associated with the disease of the one or more users. The AI engine may be further configured tomap, by using the trained model, the one or more symptoms with a set of parameters, the set of parameters may be determined by searching a medical knowledgebase for similarity of the set of parameters with the one or more symptoms. The medical knowledgebase comprises static medical resources, medical databases, online medical resources, and peer reviewed journals for a target disease. The AI engine may be then configured to predict, the disease based on the mapped one or more symptoms with the set of parameters associated with the target disease. The system may then be configured to facilitate a final response corresponding to the predicted disease to the one or more users.

In an embodiment, the respective response may be mapped with the information service and may be transmitted in real-time to the plurality of first computing devices.

In an embodiment, the knowledge graph design may be configured to model one or more dependencies associated with one or more conditional queries to be received from the one or more users.

In an embodiment, the first set of attributes further may include a dialogue state, an utterance state, a patient state, a disease state, and a symptom state, wherein the dialogue state comprises a conversation between the user and the system (110). The utterance state may include one or more exchanges that occur between the user and the system, the patient state may include a complete information of the user, a plurality of responses to the set of input queries, the disease state may include a complete information about a target disease and the symptom state may include information about a plurality of symptoms associated with the target disease.

In an embodiment, the AI engine may be further configured to predict the disease based on a scoring mechanism that comprises any or a combination of differential diagnosis of the Patient State, a symptom coverage score associated with a coverage of the one or more symptoms expressed by the user corresponding to the disease, a Relative Disease Score pertaining to relevance of the disease compared with other diseases and an Attribute Mismatch Decay score pertaining to a mismatch of the first set of attributes with the second set of attributes.

In an embodiment, the AI engine may be further configured to predict the disease based on a plurality of contexts such as any or a combination of disease priors, risk factors, lab-tests, body organs, body systems.

In an embodiment, the AI engine may be further configured to provide an automatic learning of one or more weights and the set of parameters from the knowledge graph and a feedback analysis to self-improve prediction of the disease and update the knowledge graph based on a revised one or more weights in the knowledge graph as suggested by a plurality of medical experts.

In an embodiment, the system may be further configured to collect a plurality of points of data chosen by a plurality of medical experts to gather, refine and enrich the medical knowledgebase.

In an embodiment, an interface unit associated with the plurality of first computing devices may be configured to display a plurality of information associated with the predicted disease.

In an embodiment, the AI engine may be further configured to obtain any or a combination of a previous utterance and a response by the user for the previous utterance question for retrieval of information of a user from the dialogue state. The previous utterance question may be derived by the system from the received set of input queries. The AI engine may be configured to obtain one or more user answered symptoms from the symptom state, obtain a demography of the user from the patient state and further obtain, from the disease state, a list of topmost diseases that relate with the one or more symptoms of the user with highest probability.

In an embodiment, the AI engine may be further configured to understand the previous utterance to decide the next utterance based on any or a combination of a previous demographic utterance response, a previous symptom utterance response, and a previous attribute response.

In an embodiment, the AI engine may be further configured to filter, unrelated one or more symptoms from the knowledge graph, determine one or more highly likely diseases based on the one or more symptoms of the user, the scores associated with the one or more symptom associated with the disease; and, predict, an actual disease based on the filtering of unrelated one or more symptoms and determination the one or more highly likely diseases.

In an aspect, the present disclosure provides for a user equipment (UE) facilitating a response of prediction of a disease. The UE may include a processor and a receiver operatively coupled to a plurality of first computing devices, the processor coupled with a memory that stores instructions which when executed by the processor causes the UE to receive a set of input queries from one or more users associated with the plurality of first computing devices, the set of input queries pertaining to one or more symptoms associated with a disease. The UE may be further configured to extract a first set of attributes from the received set of input queries, the first set of attributes pertaining to the one or more symptoms of the disease and then extract a second set of attributes from the received set of input queries, the second set of attributes pertaining to demographic conditions and location. Based on the extracted first and second attributes, the UE may be further configured to generate, by using an artificial intelligence (AI) engine, a trained model configured to process each input query and based on the trained model, the UE may be configured to map, by using the AI engine, each input query with a respective response that may be associated with an information service that may be further associated with a knowledge graph comprising interconnections between a target disease and the one or more symptoms associated with the disease of the one or more users. The AI engine may be further configured to map, by using the trained model, the one or more symptoms with a set of parameters, the set of parameters may be determined by searching a medical knowledgebase for similarity of the set of parameters with the one or more symptoms. The medical knowledgebase comprises static medical resources, medical databases, online medical resources, and peer reviewed journals for a target disease. The AI engine may be then configured to predict, the disease based on the mapped one or more symptoms with the set of parameters associated with the target disease. The UE may then be configured to facilitate a final response corresponding to the predicted disease to the one or more users.

In an aspect, the present disclosure provides for a method for facilitating a response of prediction of a disease. The method may include the step of receiving, by one or more processors, a set of input queries from one or more users associated with the plurality of first computing devices, the set of input queries pertaining to one or more symptoms associated with a disease. The one or more processors may be operatively coupled to a plurality of first computing devices and may be coupled with a memory that stores instructions which may be executed by the one or more processors. The method may further include the steps of extracting, by the one or more processors, a first set of attributes from the received set of input queries, the first set of attributes pertaining to the one or more symptoms of the disease and then the step of extracting, by the one or more processors, a second set of attributes from the received set of input queries, the second set of attributes pertaining to demographic conditions and location. Based on the extracted first and second attributes, the method may further include the step of generating, by using an artificial intelligence (AI) engine, a trained model configured to process each input query, the AI engine may be associated with the one or more processors. Based on the trained model, the method may further include the step of mapping, by using the AI engine, each input query with a respective response that may be associated with an information service that may be further associated with a knowledge graph comprising interconnections between a target disease and the one or more symptoms associated with the disease of the one or more users. The method may further include the steps of mapping, by using the trained model by the AI engine, the one or more symptoms with a set of parameters determined by searching a medical knowledgebase for similarity of the set of parameters with the one or more symptoms. The medical knowledgebase may include static medical resources, medical databases, online medical resources, and peer reviewed journals for a target disease. The method may then include the step of predicting, by the AI engine, the disease based on the mapped one or more symptoms with the set of parameters associated with the target disease. Furthermore, the method may include the step of facilitating, by the one or more processors, a final response corresponding to the predicted disease to the one or more users.

### BRIEF DESCRIPTION OF DRAWINGS

The accompanying drawings, which are incorporated herein, and constitute a part of this invention, illustrate exemplary embodiments of the disclosed methods and systems in which like reference numerals refer to the same parts throughout the different drawings. Components in the drawings are not necessarily to scale, emphasis instead being placed upon clearly illustrating the principles of the present invention. Some drawings may indicate the components using block diagrams and may not represent the internal circuitry of each component. It will be appreciated by those skilled in the art that invention of such drawings includes the invention of electrical components, electronic components or circuitry commonly used to implement such components.

FIG. 1 illustrates an exemplary network architecture (100) in which or with which the system of the present disclosure can be implemented, in accordance with an embodiment of the present disclosure.

FIG. 2A illustrates an exemplary representation (200) of system (110), in accordance with an embodiment of the present disclosure.

FIG. 2B illustrates an exemplary representation (250) of user equipment (UE) 108), in accordance with an embodiment of the present disclosure.

FIG. 3 illustrates an exemplary method flow diagram (300) depicting a method for in accordance with an embodiment of the present disclosure.

FIG. 4 illustrates an exemplary block flow representation (400) of the proposed system depicting the memory states and utterances cycle in the dialog flow, in accordance with an embodiment of the present disclosure.

FIG. 5 illustrates an exemplary block diagram representation (500) of a disease prediction model, in accordance with an embodiment of the present disclosure.

FIGS. 6A-6C illustrate exemplary representations of flow diagram that elaborate upon the proposed modules of the proposed disease prediction method and system, in accordance with an embodiment of the present disclosure.

FIG. 7 illustrates an exemplary computer system in which or with which embodiments of the present invention can be utilized in accordance with embodiments of the present disclosure. The foregoing shall be more apparent from the following more detailed description of the invention.

### BRIEF DESCRIPTION OF INVENTION

In the following description, for the purposes of explanation, various specific details are set forth in order to provide a thorough understanding of embodiments of the present disclosure. It will be apparent, however, that embodiments of the present disclosure may be practiced without these specific details. Several features described hereafter can each be used independently of one another or with any combination of other features. An individual feature may not address all of the problems discussed above or might address only some of the problems discussed above. Some of the problems discussed above might not be fully addressed by any of the features described herein.

The present invention provides solution to the above-mentioned problem in the art by providing a system and a method for an efficient and adaptive disease prediction system as a part of an expert-based artificial intelligence (AI) symptom-checker. The symptom checker is purpose-built for the Indian demography with expert curated medical knowledge and a knowledge-graph driven AI reasoning engine. The system may diagnose a wide variety of diseases accurately in an under 3-minute chatbot based conversation. The system architecture comprises of state-based components namely dialogue, patient, utterance, disease and symptom state. The disease prediction module may use a complex knowledge graph coupled with the state information to predict disease scores, predict disease scores based on various factors such as symptom coverage, relative disease importance, diseases priors, risk factors, lab tests, body organs and body systems and further provide mechanisms to arrive at a convergence based on factors such as confidence scores, questions asked and the like.

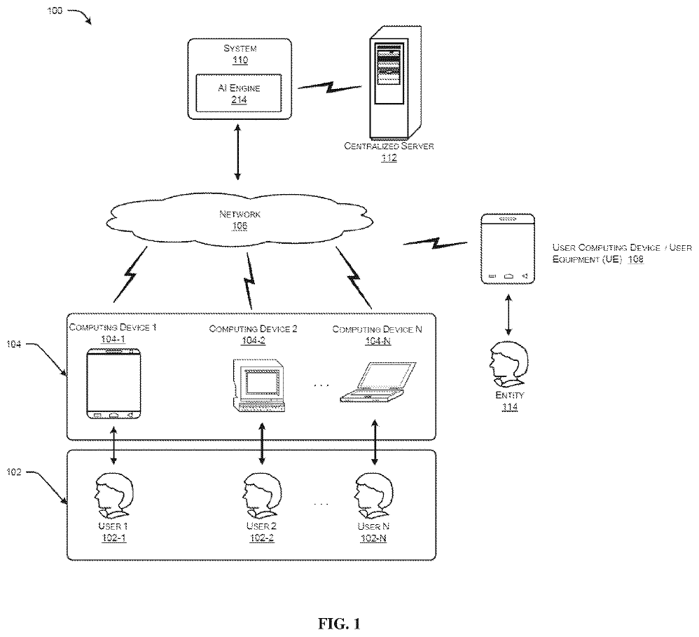

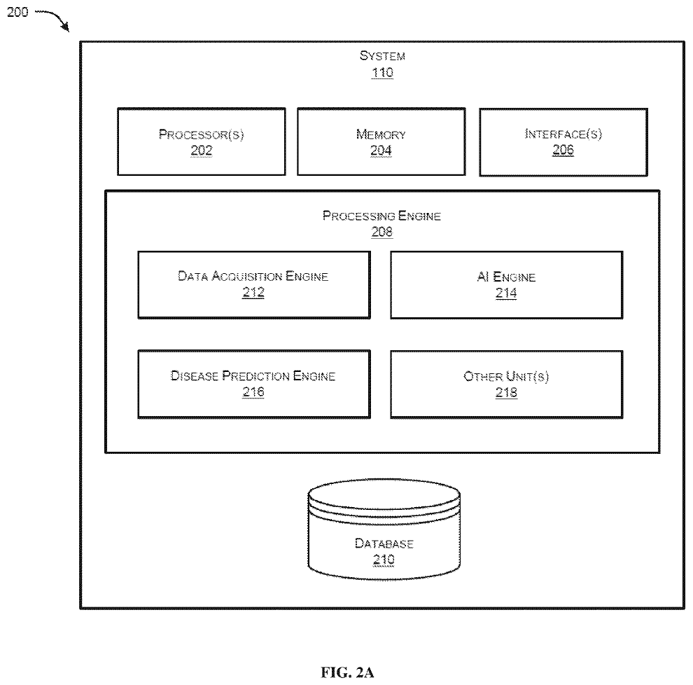

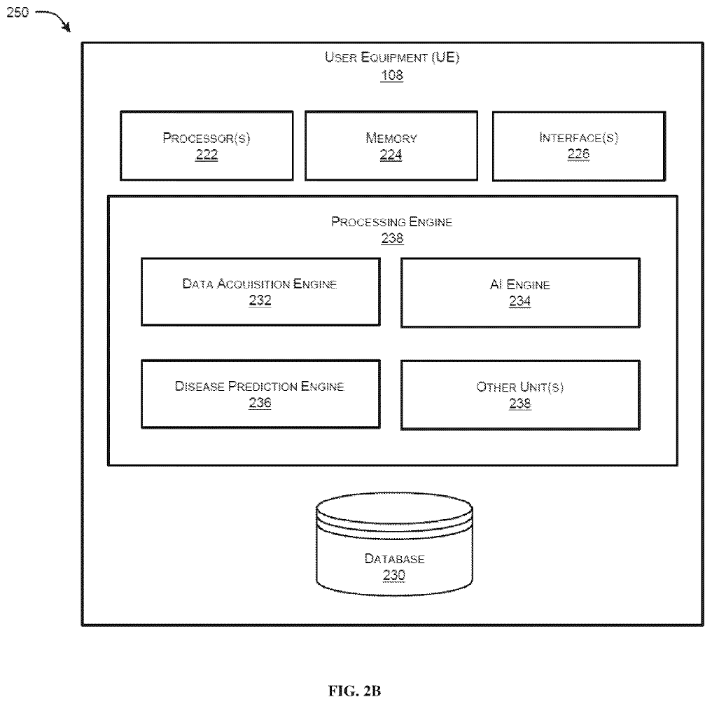

Referring to FIG. 1 that illustrates an exemplary network architecture (100) in which or with which system (110) of the present disclosure can be implemented, in accordance with an embodiment of the present disclosure. As illustrated, the exemplary architecture (100) includes a system (110) equipped with an artificial intelligence (AI) engine (214) (Ref. FIG. 2A) for facilitating a response of prediction of a disease to a plurality of users (102-1, 102-2, . . . 102-n) (hereinafter interchangeably referred as user; and collectively referred to as users 102 or patients (102)). Each user may be associated with at least one computing device (104-1, 104-2, . . . 104-n) (hereinafter interchangeably referred as a smart computing device or audio device; and collectively referred to as 104). The users (102) may interact with the system (110) by using their respective computing device (104), wherein the computing device (104) and the system (110) may communicate with each other over a network (106). The system (110) may be associated with a centralized server (112). Examples of the computing devices (104) can include, but are not limited to, a computing device (104) associated with healthcare and hospital-based assets, a smart phone, a portable computer, a personal digital assistant, a handheld phone and the like.

The system (110) may be further operatively coupled to a second computing device (108) (also referred to as the user computing device or user equipment (UE) hereinafter) associated with an entity (114). The entity (114) may include a company, a hospital, an organisation, a university, a lab facility, a business enterprise, or any other secured facility associated with health care research and related functionalities. In some implementations, the system (110) may also be associated with the UE (108). The UE (108) can include a handheld device, a smart phone, a laptop, a palm top and the like. Further, the system (110) may also be communicatively coupled to the one or more first computing devices (104) via a communication network (106).

Further, the network (106) can be a wireless network, a wired network, a cloud or a combination thereof that can be implemented as one of the different types of networks, such as Intranet, BLUETOOTH, MQTT Broker cloud, Local Area Network (LAN), Wide Area Network (WAN), Internet, and the like. Further, the network 106 can either be a dedicated network or a shared network. The shared network can represent an association of the different types of networks that can use variety of protocols, for example, Hypertext Transfer Protocol (HTTP), Transmission Control Protocol/Internet Protocol (TCP/IP),

Wireless Application Protocol (WAP), and the like. In an exemplary embodiment, the network 104 can be anHC-05 Bluetooth module which is an easy to use Bluetooth SPP (Serial Port Protocol) module, designed for transparent wireless serial connection setup.

According to various embodiments of the present disclosure, the system 100 can provide for an Artificial Intelligence (AI) based automatic medical attribute detection, identification and input generation by using signal processing analytics. In an illustrative embodiment, the AI techniques can include, but not limited to, any or a combination of machine learning (referred to as ML hereinafter), deep learning (referred to as DL hereinafter) using concepts of neural network techniques.

In an aspect, the system (110) can receive a set of input queries from one or more users associated with the plurality of first computing devices. For example, the set of input queries may pertain to one or more symptoms associated with a disease. The system (110) may be configured to extract a first set of attributes and second set of attributes from the received set of input queries. For example, the first set of attributes may pertain to the one or more symptoms of the disease and the second set of attributes may pertain to demographic conditions and location. Based on the extracted first and second attributes, a trained model may be generated, by using an artificial intelligence (AI) engine (214), to process each input query. Based on the trained model, the AI engine (214) may be configured to map each input query with a respective response. The respective response may be associated with an information service, that may be further associated with a knowledge graph comprising interconnections between a target disease and the one or more symptoms associated with the disease of the one or more users. The AI engine (214) may be further configured to map, by using the trained model, the one or more symptoms with a set of parameters. The set of parameters may be determined by searching a medical knowledgebase for similarity of the set of parameters with the one or more symptoms. The medical knowledgebase may include static medical resources, medical databases, online medical resources, peer reviewed journals and the like for a target disease. The AI engine (214) may be configured to predict, the disease based on the mapped one or more symptoms with the set of parameters associated with the target disease. The system (110) may finally facilitate a final response corresponding to the predicted disease to the one or more users.

In an exemplary embodiment, the disease prediction engine (216) may use the working-memory knowledge graph that constitutes the interconnections between the primary entity nodes in the form of curated weighted edges.

In an exemplary embodiment, the working-memory knowledge graph design may allow for modelling dependencies between symptom attributes and symptom-attribute values further providing a possibility to ask conditional questions.

In an exemplary embodiment, the first set of attributes may further include a dialogue state, a patient state, a disease state, symptom state and an utterance state. The dialogue state may orchestrate a conversation between the patient (102) and the system (110) by keeping the knowledge of all states. The utterance state may include exchanges that occur between the user and the system such as a response from the user and Question from the system (110). The patient state which contains a complete information that the patient has shared with us, including but not limited to age, gender, dominating/bothering symptoms, responses to the questions asked. Patient state evolves over time, getting updated as and when user responses to the questions. The symptom state may contain the information about the various symptoms, attribute information and their possible values. The disease state may include a complete information about the disease, including but not limited to age, gender, risk factors, body system, consultation specializations, and the like.

In an exemplary embodiment, the AI engine (214) may include a scoring mechanism for differential diagnosis from the above-mentioned Patient State. The scoring mechanism may further include a symptom coverage score that may be associated with coverage of the symptoms expressed by the patient to that of the diseases. The scoring mechanism may also include a Relative Disease Score that may pertain to relevance of the disease score when compared with other diseases and an Attribute Mismatch Decay that pertain to mismatch of the categorical attributes. For example, a mismatch between a Dry Cough and a Wet Cough.

In an exemplary embodiment, the system (110) may provide a schema design allows for factoring in other contexts such as disease priors, risk factors, lab-tests, body organs, body systems and the like and hence more intelligent disease prediction model based on various medical contexts.

In an exemplary embodiment, the AI engine (214) may provide an automatic learning of weights and parameters from knowledge graph and feedback system to self-improve the disease prediction and a mechanism for accommodating the revised weights in the knowledge graph as suggested by experts.

In an exemplary embodiment, the system (110) may be configured to collect various points of data chosen by a plurality of medical experts to gather, refine and enrich the medical knowledgebase.

In an exemplary embodiment, a retrieval of patient's information from the dialogue state may include the steps of getting a previous utterance question derived by the system and patient's response for the utterance question, getting the user answered symptoms from the symptom state, getting the patient's demographics like age and gender from the patient state and getting the top n diseases with highest probability from the disease state.

In an exemplary embodiment, the AI engine (214) may be configured to understand the previous utterances to decide the next utterance by a decision making based on any or a combination of previous demographic utterance response, previous symptom utterance response, previous attribute response and the like.

In an exemplary embodiment, the disease prediction engine (216) may relate the disease state by finding the highly likely diseases based on the present symptoms, the weights of symptom associated with the disease and the derived symptom and by filtering the symptoms mapped to likely diseases based on age and gender.

FIG. 2A illustrates an exemplary representation (200) of system (110), in accordance with an embodiment of the present disclosure.

In an aspect, the system (110) may comprise one or more processor(s) (202). The one or more processor(s) (202) may be implemented as one or more microprocessors, microcomputers, microcontrollers, digital signal processors, central processing units, logic circuitries, and/or any devices that process data based on operational instructions. Among other capabilities, the one or more processor(s) (202) may be configured to fetch and execute computer-readable instructions stored in a memory (204) of the system (110). The memory (204) may be configured to store one or more computer-readable instructions or routines in a non-transitory computer readable storage medium, which may be fetched and executed to create or share data packets over a network service. The memory (204) may comprise any non-transitory storage device including, for example, volatile memory such as RAM, or non-volatile memory such as EPROM, flash memory, and the like.

In an embodiment, the system (102) may include an interface(s) 206. The interface(s) 206 may comprise a variety of interfaces, for example, interfaces for data input and output devices, referred to as I/O devices, storage devices, and the like. The interface(s) 206 may facilitate communication of the system (102). Examples of such components include, but are not limited to, processing engine(s) 208 and a database 210.

The processing engine(s) (208) may be implemented as a combination of hardware and programming (for example, programmable instructions) to implement one or more functionalities of the processing engine(s) (208). In examples described herein, such combinations of hardware and programming may be implemented in several different ways. For example, the programming for the processing engine(s) (208) may be processor executable instructions stored on a non-transitory machine-readable storage medium and the hardware for the processing engine(s) (208) may comprise a processing resource (for example, one or more processors), to execute such instructions. In the present examples, the machine-readable storage medium may store instructions that, when executed by the processing resource, implement the processing engine(s) (208). In such examples, the system (110) may comprise the machine-readable storage medium storing the instructions and the processing resource to execute the instructions, or the machine-readable storage medium may be separate but accessible to the system (110) and the processing resource. In other examples, the processing engine(s) (208) may be implemented by electronic circuitry.

The processing engine (208) may include one or more engines selected from any of a data acquisition (212), an AI engine (214), a disease prediction engine (216) and other units (218).

In an embodiment, the data acquisition (212) can receive a set of input queries from one or more users associated with the plurality of first computing devices. For example, the set of input queries may pertain to one or more symptoms associated with a disease. The AI engine (214) may extract a first set of attributes and second set of attributes from the received set of input queries. For example, the first set of attributes may pertain to the one or more symptoms of the disease and the second set of attributes may pertain to demographic conditions and location. Based on the extracted first and second attributes, a trained model may be generated, by using the AI engine (214), to process each input query. Based on the trained model, the AI engine (214) may be configured to map each input query with a respective response. The respective response may be associated with an information service, that may be further associated with a knowledge graph comprising interconnections between a target disease and the one or more symptoms associated with the disease of the one or more users. The AI engine (214) may be further configured to map, by using the trained model, the one or more symptoms with a set of parameters. The set of parameters may be determined by searching a medical knowledgebase for similarity of the set of parameters with the one or more symptoms. The medical knowledgebase may include static medical resources, medical databases, online medical resources, peer reviewed journals and the like for a target disease. The disease prediction engine (216) may be configured to predict, the disease based on the mapped one or more symptoms with the set of parameters associated with the target disease. The AI engine (214) may finally facilitate a final response corresponding to the predicted disease to the one or more users.

In an exemplary embodiment, the disease prediction engine (216) may use the working-memory knowledge graph that constitutes the interconnections between the primary entity nodes in the form of curated weighted edges to predict the disease.

FIG. 2B illustrates an exemplary representation (250) of the user equipment (UE) (108), in accordance with an embodiment of the present disclosure. In an aspect, the UE (108) may comprise a processor (222). The more processor (222) may be implemented as one or more microprocessors, microcomputers, microcontrollers, digital signal processors, central processing units, logic circuitries, and/or any devices that process data based on operational instructions. Among other capabilities, the processor(s) (222) may be configured to fetch and execute computer-readable instructions stored in a memory (224) of the UE (108). The memory (224) may be configured to store one or more computer-readable instructions or routines in a non-transitory computer readable storage medium, which may be fetched and executed to create or share data packets over a network service. The memory (224) may comprise any non-transitory storage device including, for example, volatile memory such as RAM, or non-volatile memory such as EPROM, flash memory, and the like.

In an embodiment, the UE (108) may include an interface(s) 206. The interface(s) 206 may comprise a variety of interfaces, for example, interfaces for data input and output devices, referred to as I/O devices, storage devices, and the like. The interface(s) 206 may facilitate communication of the UE (108). Examples of such components include, but are not limited to, processing engine(s) 228 and a database (230).

The processing engine(s) (228) may be implemented as a combination of hardware and programming (for example, programmable instructions) to implement one or more functionalities of the processing engine(s) (228). In examples described herein, such combinations of hardware and programming may be implemented in several different ways. For example, the programming for the processing engine(s) (228) may be processor executable instructions stored on a non-transitory machine-readable storage medium and the hardware for the processing engine(s) (228) may comprise a processing resource (for example, one or more processors), to execute such instructions. In the present examples, the machine-readable storage medium may store instructions that, when executed by the processing resource, implement the processing engine(s) (228). In such examples, the UE (108) may comprise the machine-readable storage medium storing the instructions and the processing resource to execute the instructions, or the machine-readable storage medium may be separate but accessible to the UE (108) and the processing resource. In other examples, the processing engine(s) (228) may be implemented by electronic circuitry.

The processing engine (228) may include one or more engines selected from any of a data acquisition (232), an AI engine (234), a disease prediction engine (236) and other units (238).

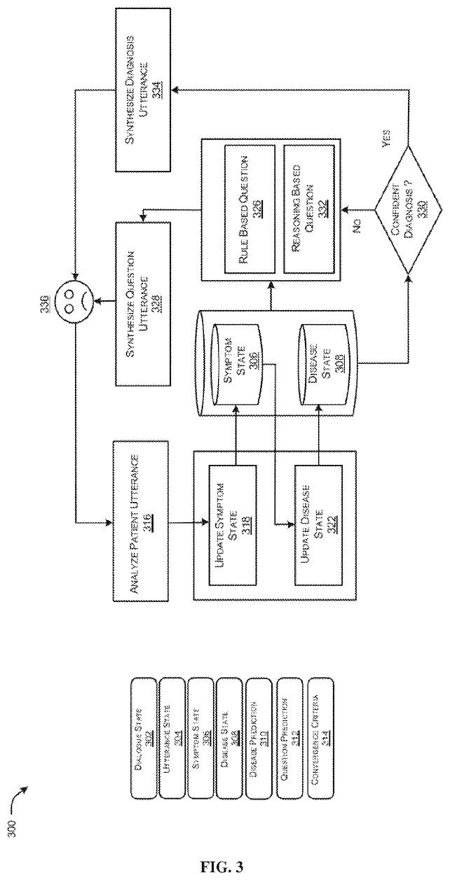

FIG. 3 illustrates an exemplary block diagram (300) representation of the proposed system in accordance with an embodiment of the present disclosure. In an exemplary embodiment, the system (110) may include dialogue state module (302) that may be configured to orchestrate the conversation between Patient and AI-Doctor system by keeping the knowledge of all states.

The system (110) may include an utterance state module (304) may include exchanges that occur between the user and the system such as a response from the user and question from the system. The system (110) may a symptom state module (306) that may include representing symptom-state in terms of presence/absence and attributes. The system (110) may further include a disease state module (308) that may determine probability of whether a disease is present or not present given symptoms. The system (110) may include a disease prediction module (310) that may determine given the current set of symptoms what is the probability of each disease. The system (110) may include a question prediction module (312) that may predict, given the symptom and disease state what is the next best question to ask. The system (110) may analyze patient utterance (316) and a symptom state may be updated (318) and send it to a symptom state module (306) output of which may be sent to update a disease state (322) and stored in disease state module (308). If diagnosis is not confident (330) then reasoning (332) based question may be provided else if diagnosis is confident (330) then synthesize diagnosis utterance (334). The output from symptom state and disease state may be provided to a rule-based question module (326) to synthesize question utterance (336).

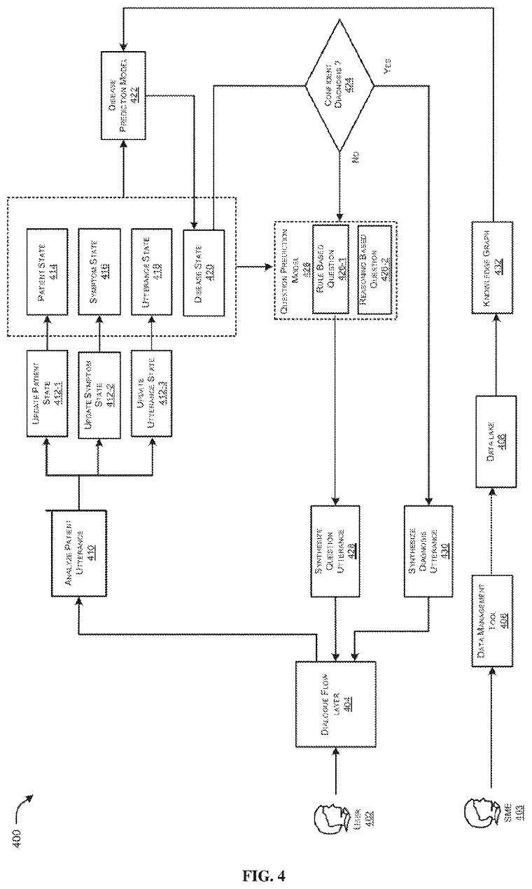

FIG. 4 illustrates an exemplary flow diagram (400) representation of the proposed method in accordance with an embodiment of the present disclosure. The method may include a dialogue flow layer (404) between the user (402) and the system (110) that may analyze the patient utterance (410) from which the demographics, symptoms and utterances of the patient (412) are provided to the patient state (414), symptom state (416), utterance state (418), disease state (420). The disease prediction model (422) is then triggered which internally uses the updated patient state, utterance state and symptom state. If diagnosis is not confident (424) then rule or reasoning (426) based question may be provided else if diagnosis is confident (424) then synthesize diagnosis utterance (430). The question prediction model (426) internally uses the updated patient state, utterance state and symptom state to synthesize question utterance (428). The data can also be provided from an SME (403) which is then sent to a management tool (406) then to the data lake (408), then to the knowledge graph (432) and then to the disease prediction model (422).

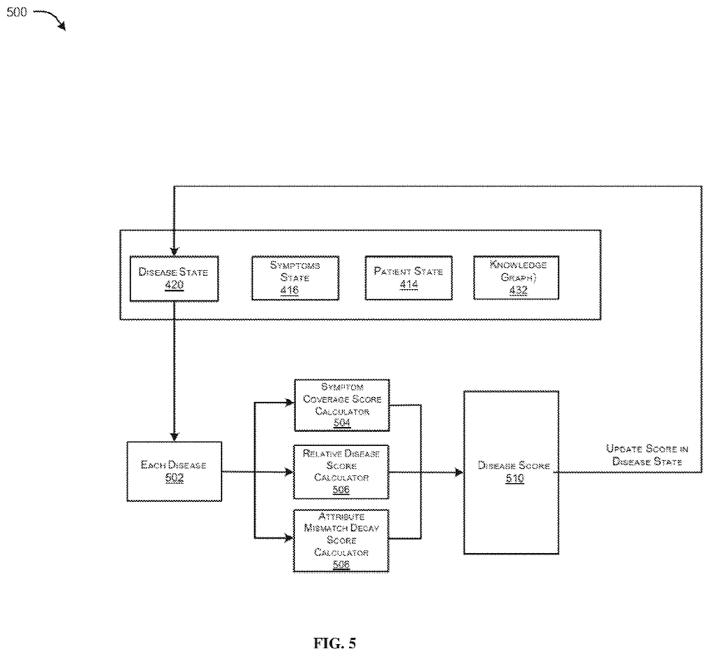

FIG. 5 illustrates an exemplary block diagram representation of a disease prediction model (500), in accordance with an embodiment of the present disclosure. In an exemplary embodiment, the disease prediction model may include a scoring mechanism for differential diagnosis from the Patient State (414). The scoring mechanism may further include a symptom coverage score (504) that may be associated with coverage of the symptoms expressed by the patient to that of the diseases. The scoring mechanism may also include a Relative Disease Score (506) that may pertain to relevance of the disease score when compared with other diseases and an Attribute Mismatch Decay (506) that pertain to mismatch of the categorical attributes. For example, a mismatch between a Dry Cough and a Wet Cough. A disease score (510) may be generated based on the scores obtained from the combination of the symptom coverage score (504), Relative Disease Score (506) and the Attribute Mismatch Decay (506). The disease score determined is updated and stored in the disease state (420) and thereby the knowledge graph (432) may be generated.

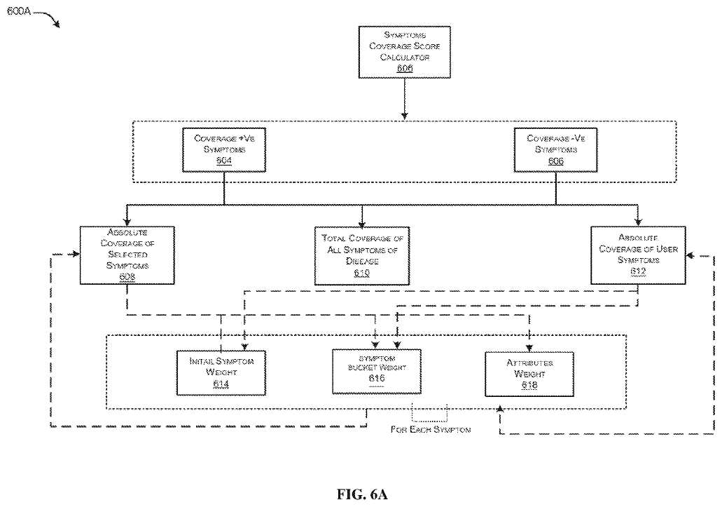

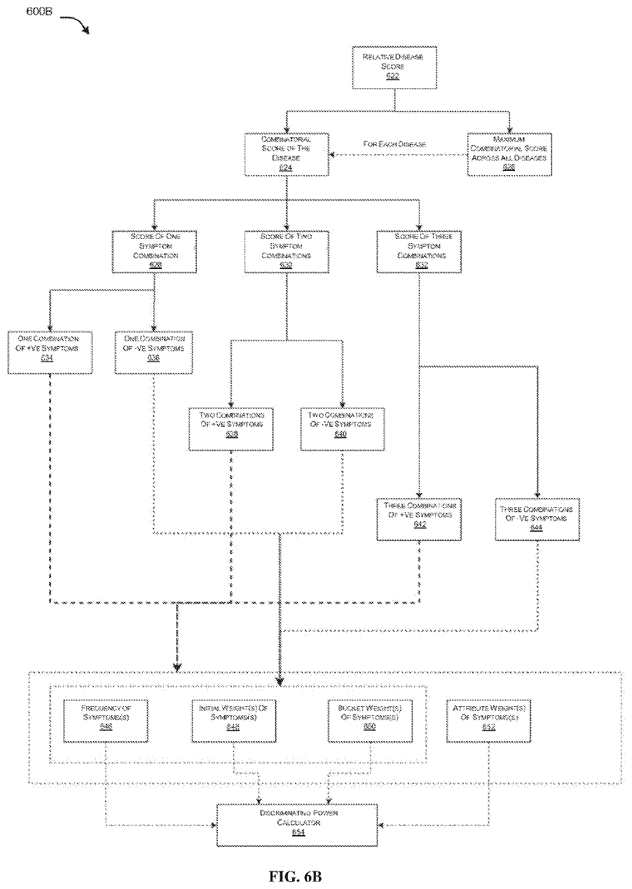

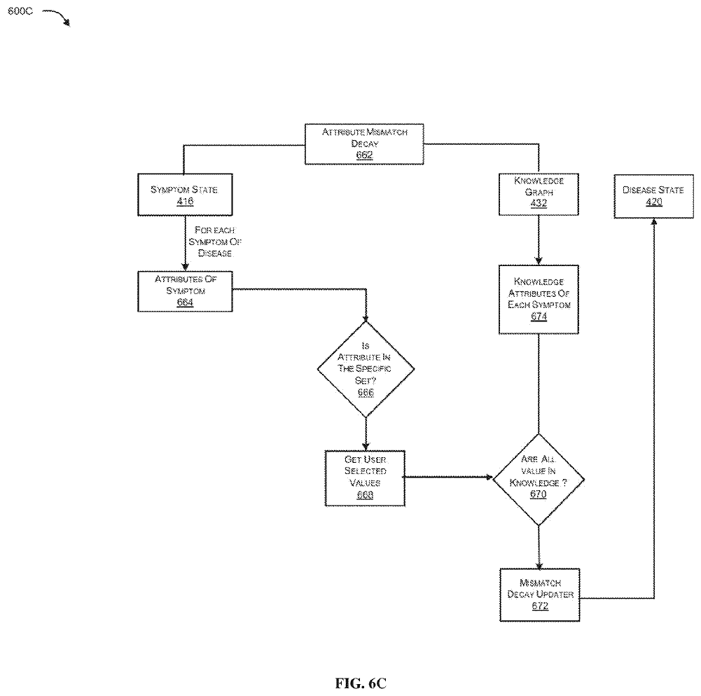

FIGS. 6A-6C illustrate exemplary representations of flow diagram that elaborate upon the proposed method and system, in accordance with an embodiment of the present disclosure. As illustrated in FIG. 6A, in an aspect, the method for symptom coverage score generation (600) may provide coverage of the symptoms expressed by the patient to that of the disease and may include a symptom coverage score calculator (602). The symptom coverage score calculator (602) may further include a coverage of positive symptoms (604) that involves a score given to the coverage of the symptoms which are present in the disease and are answered affirmative by the patient. The symptom coverage score calculator (602) may also include a Coverage of Negative symptoms (606) that involves a penalty score given to the coverage of the symptoms which are present in the disease and are answered negative by the patient. The symptom coverage score calculator (602) may provide an Initial Symptom weight (614), a symptom disease weight (616), and an attribute weight (618) with the help of dominating symptoms with which the patient comes to the symptom are given higher importance such as absolute coverage of user selected symptoms (608), total coverage of all symptoms of the disease (610) and absolute coverage of the user symptoms.

FIG. 6B illustrates a Relative Disease Score method (620) that provides a relevance of the disease score when compared with other diseases and may include a relative disease score generator (622) that provides any or a combination of a Combinational Score the disease (624) that may consider different combinations of the symptoms and the maximum score of the combinations (626) is taken as the final score. For example, the combinational Score the disease (624) may consider a score of one symptom combinations (628), a score of two symptom combinations (630), a score of three symptom combinations (632). The score of one symptom combinations (628) may include one combination of positive symptom and one combination of negative symptom. The score of two symptom combinations (630) may include two combination of positive symptoms and two combination of negative symptoms. The score of three symptom combinations (632) may include three combination of positive symptoms and three combination of negative symptoms and so on. Then a discriminating power calculator (654) may calculate a uniqueness of the symptom the following factors:
Frequency of the symptoms (646): Frequency is inversely proportional to the importance or the discriminating power, I.e. higher frequency means less importance and vice-versa
Initial weights of the symptoms (648): Higher importance is given to the dominating symptoms
Bucket weights of the symptoms (650): Weights from the knowledge graph is considered here
Attribute weights of the symptoms (652): Attribute weights from the knowledge graph is considered here

FIG. 6C illustrates an attribute mismatch generation method (660) that may determined amismatch of the categorical attributes. The method (660) includes the step of checking attribute mismatch decay (662) by comparing the symptom state (416) and the knowledge graph (432). For each symptom of the disease, attributes of the symptom (664) may be checked for a specific attribute (666). If the attribute is specific then get user selected values (668). If all the user selected values are not in knowledge (670), update the mismatch score (672) and then provide to disease state (420). For example, if the disease has specific attribute of the symptom and the patient has different one, then a mismatch decay is added to the scoring, this helps in eliminating the E.g.: Dry Cough vs Wet Cough.

In an exemplary embodiment, the ultimate step of disease prediction and converging the triage with confidence always works based on the convergence criteria which has multiple validations and policies. The input parameters may include the disease state, the Symptom state, the convergence parameters. In a way of example and not as a limitation, convergence criteria may be obtained by considering:
a. Minimum symptoms that should be asked=3
b. Maximum symptoms that can be asked=10
c. Relative threshold for confusion diseases=0.8
d. Top disease score threshold=0.8
In an exemplary embodiment, various set of policies have a defined order to follow in the validation process as shown below
if (NumberOfSymptomsAsked(s)<min_symptoms_to_ask(p))
   return false
if (NumberOfSymptomsAsked(s)>max_symptoms_to_ask(p))
   return true
if (!AllAttributesOfInitialSymptoms(s))
   return false
if (!AllBucket1SymptomsOfTopConfusionDiseases(s, d, p))
   return false
if (!AllAttributesOfBucket1PresentSymptomsOfTopDisease(s, d))
   return false
if (ScoreOfHighestDisease (d)>confidence_threshold(p))
   return true
return false

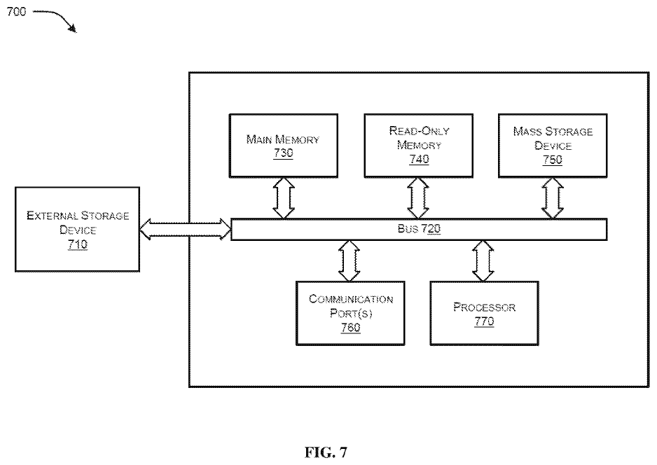

FIG. 7 illustrates an exemplary computer system in which or with which embodiments of the present invention can be utilized in accordance with embodiments of the present disclosure. As shown in FIG. 7, computer system 700 can include an external storage device 710, a bus 720, a main memory 730, a read only memory 740, a mass storage device 770, communication port 760, and a processor 770. A person skilled in the art will appreciate that the computer system may include more than one processor and communication ports. Processor 770 may include various modules associated with embodiments of the present invention. Communication port 760 may be chosen depending on a network, or any network to which computer system connects. Memory 730 can be Random Access Memory (RAM), or any other dynamic storage device commonly known in the art. Read-only memory 740 can be any static storage device(s). Mass storage 770 may be any current or future mass storage solution, which can be used to store information and/or instructions.

Bus 720 communicatively couples processor(s) 770 with the other memory, storage and communication blocks.

Optionally, operator and administrative interfaces, e.g. a display, keyboard, joystick and a cursor control device, may also be coupled to bus 720 to support direct operator interaction with a computer system. Other operator and administrative interfaces can be provided through network connections connected through communication port 760. Components described above are meant only to exemplify various possibilities. In no way should the aforementioned exemplary computer system limit the scope of the present disclosure.

Thus, the present disclosure provides a unique and inventive solution for a diagnostics-as-a-service that may include a medical knowledge has been curated by the medical experts keeping the Indian demography in mind. The system may benefit healthcare by collectively increasing efficiency, providing quick diagnostics to less privileged patients, and reduce overall costs and serve as a reference to medical students assisting them in carrying out a triage with the patient and diagnose accurately.

While considerable emphasis has been placed herein on the preferred embodiments, it will be appreciated that many embodiments can be made and that many changes can be made in the preferred embodiments without departing from the principles of the invention. These and other changes in the preferred embodiments of the invention will be apparent to those skilled in the art from the disclosure herein, whereby it is to be distinctly understood that the foregoing descriptive matter to be implemented merely as illustrative of the invention and not as limitation.

A portion of the disclosure of this patent document contains material which is subject to intellectual property rights such as, but are not limited to, copyright, design, trademark, IC layout design, and/or trade dress protection, belonging to Jio Platforms Limited (JPL) or its affiliates (herein after referred as owner). The owner has no objection to the facsimile reproduction by anyone of the patent document or the patent disclosure, as it appears in the Patent and Trademark Office patent files or records, but otherwise reserves all rights whatsoever. All rights to such intellectual property are fully reserved by the owner.

### ADVANTAGES OF THE PRESENT DISCLOSURE

The present disclosure provides for a system and a method that facilitates retrieval of patient's personal and demographic information such as age and gender for better filtering on possible diagnosis.

The present disclosure provides for a working-memory knowledge graph design allows for modelling dependencies between symptom attributes and symptom-attribute values further providing a possibility to ask conditional questions.

The present disclosure provides for a Patient State monitoring module which contains complete information that the patient has shared with us, including but not limited to age, gender, dominating/bothering symptoms, responses to the questions asked.

The present disclosure provides for a scoring mechanism for differential diagnosis from the above-mentioned Patient State.

The present disclosure provides for a diagnostics-as-a-service that render immediate online diagnostic solutions to the people, even to the remotest areas of the Indian populace.

The present disclosure provides for a diagnostics-as-a-service that assist medical students to learn differential diagnosis.

The present disclosure provides for a diagnostics-as-a-service that presenting itself as a flagship product.

We claim:

1. A system (110) for facilitating a response of prediction of a disease, said system (110) comprising;
one or more processors (202) operatively coupled to a plurality of first computing devices (104), the one or more processors (202) coupled with a memory (204), wherein said memory (204) stores instructions which when executed by the one or more processors (202) causes said system (110) to:
receive a set of input queries from one or more users associated with the plurality of first computing devices, said set of input queries pertaining to one or more symptoms associated with a disease;
extract a first set of attributes from the received set of input queries, wherein the first set of attributes pertain to the one or more symptoms of the disease;
extract a second set of attributes from the received set of input queries, the second set of attributes pertaining to demographic conditions and location;
based on the extracted first and second attributes, generate, by using an artificial intelligence (AI) engine (214), a trained model configured to process each input query;
based on the trained model, map, by using the AI engine (214), each said input query with a respective response, wherein said respective response is associated with an information service, and wherein the information service is further associated with a knowledge graph comprising interconnections between a target disease and the one or more symptoms associated with the disease of the one or more users, wherein the AI engine (214) is further configured to:
map, by using the trained model, the one or more symptoms with a set of parameters, wherein the set of parameters are determined by searching a medical knowledgebase for similarity of the set of parameters with the one or more symptoms, wherein the medical knowledgebase comprises static medical resources, medical databases, online medical resources, and peer reviewed journals for a target disease; and,
predict, the disease based on the mapped one or more symptoms with the set of parameters associated with the target disease; and facilitate a final response corresponding to the predicted disease to the one or more users.

2. The system as claimed in claim 1, wherein said respective response is mapped with the information service and is transmitted in real-time to the plurality of first computing devices.

3. The system as claimed in claim 1, wherein the knowledge graph design is configured to model one or more dependencies associated with one or more conditional queries to be received from the one or more users.

4. The system as claimed in claim 1, wherein the first set of attributes further comprises a dialogue state, an utterance state, a patient state, a disease state, and a symptom state, wherein the dialogue state comprises a conversation between the user (102) and the system (110), wherein the utterance state comprises one or more exchanges that occur between the user and the system, wherein the patient state comprises a complete information of the user, a plurality of responses to the set of input queries, wherein the disease state comprises a complete information about a target disease and wherein the symptom state comprises information about a plurality of symptoms associated with the target disease.

5. The system as claimed in claim 1, wherein the AI engine (214) is further configured to predict the disease based on a scoring mechanism that comprises any or a combination of differential diagnosis of the Patient State, a symptom coverage score associated with a coverage of the one or more symptoms expressed by the user corresponding to the disease, a Relative Disease Score pertaining to relevance of the disease compared with other diseases and an Attribute Mismatch Decay score pertaining to a mismatch of the first set of attributes with the second set of attributes.

6. The system as claimed in claim 1, wherein the AI engine (214) is further configured to predict the disease based on a plurality of contexts such as any or a combination of disease priors, risk factors, lab-tests, body organs, body systems.

7. The system as claimed in claim 1, wherein the AI engine (214) is configured to
provide an automatic learning of one or more weights and the set of parameters from the knowledge graph and a feedback analysis to self-improve prediction of the disease;
and update the knowledge graph based on a revised one or more weights in the knowledge graph as suggested by a plurality of medical experts.

8. The system as claimed in claim 1, wherein the system is further configured to collect a plurality of points of data chosen by a plurality of medical experts to gather, refine and enrich the medical knowledgebase.

9. The system as claimed in claim 1, wherein an interface unit associated with the plurality of first computing devices is configured to display a plurality of information associated with the predicted disease.

10. The system as claimed in claim 4, wherein the AI engine (214) is further configured to:
Obtain any or a combination of a previous utterance and a response by the user for the previous utterance question for retrieval of information of a user from the dialogue state, wherein the previous utterance question is derived by the system from the received set of input queries;
obtain one or more user answered symptoms from the symptom state;
obtain a demography of the user from the patient state;
and obtain, from the disease state, a list of topmost diseases that relate with the one or more symptoms of the user with highest probability.

11. The system as claimed in claim 10, wherein the AI engine (214) is further configured to:
understand the previous utterance to decide the next utterance based on any or a combination of a previous demographic utterance response, a previous symptom utterance response, and a previous attribute response.

12. The system as claimed in claim 10, wherein the AI engine (214) is further configured to:
Filter, unrelated one or more symptoms from the knowledge graph;
determine one or more highly likely diseases based on the one or more symptoms of the user, the scores associated with the one or more symptom associated with the disease; and,
predict, an actual disease based on the filtering of unrelated one or more symptoms and determination the one or more highly likely diseases.

13. A user equipment (UE) for facilitating a response of prediction of a disease, said UE comprising;
a processor (222) and a receiver operatively coupled to a plurality of first computing devices (104), the processor (222) coupled with a memory (224), wherein said memory (224) stores instructions which when executed by the one or more processors (222) causes said UE (108) to:
receive, by the receiver, a set of input queries from one or more users associated with the plurality of first computing devices, said set of input queries pertaining to one or more symptoms associated with a disease;
extract, by the processor (222), a first set of attributes from the received set of input queries, wherein the first set of attributes pertain to the one or more symptoms of the disease;
extract, by the processor (222), a second set of attributes from the received set of input queries, the second set of attributes pertaining to demographic conditions and location;
based on the extracted first and second attributes, generate, by using an artificial intelligence (AI) engine (234), a trained model configured to process each input query, wherein the AI engine (234) is associated with the processor (222);
based on the trained model, map, by using the AI engine (234), each said input query with a respective response, wherein said respective response is associated with an information service, and wherein the information service is further associated with a knowledge graph comprising interconnections between a target disease and the one or more symptoms associated with the disease of the one or more users, wherein the AI engine (234) is further configured to:
map, by using the trained model, the one or more symptoms with a set of parameters, wherein the set of parameters are determined by searching a medical knowledgebase for similarity of the set of parameters with the one or more symptoms, wherein the medical knowledgebase comprises static medical resources, medical databases, online medical resources, and peer reviewed journals for a target disease; and, predict, the disease based on the mapped one or more symptoms with the set of parameters associated with the target disease; and
facilitate, a final response corresponding to the predicted disease to the one or more users.

14. The UE as claimed in claim 13, wherein the UE is equipped with a user interface configured to display the input set of queries, the respective responses, the final response and a plurality of information related to the disease between the user and the UE.

15. A method for facilitating a response of prediction of a disease, said method comprising the steps of:
receiving, by the one or more processors (202), a set of input queries from one or more users associated with the plurality of first computing devices, said set of input queries pertaining to one or more symptoms associated with a disease, wherein the one or more processors (202) are operatively coupled to a plurality of first computing devices (104), the one or more processors (202) coupled with a memory (204), wherein said memory (204) stores instructions that are executed by the one or more processors;
extracting, by the one or more processors (202), a first set of attributes from the received set of input queries, wherein the first set of attributes pertain to the one or more symptoms of the disease;
extracting, by the one or more processors (202), a second set of attributes from the received set of input queries, the second set of attributes pertaining to demographic conditions and location;
based on the extracted first and second attributes, generating, by using an artificial intelligence (AI) engine, a trained model configured to process each input query, wherein the AI engine (214) is associated with the one or more processors (202);
based on the trained model, mapping, by using the AI engine (214), each said input query with a respective response, wherein said respective response is associated with an information service, and wherein the information service is further associated with a knowledge graph comprising interconnections between a target disease and the one or more symptoms associated with the disease of the one or more users, wherein the method further comprises the steps of:
mapping, by using the trained model by the AI engine (214), the one or more symptoms with a set of parameters, wherein the set of parameters are determined by searching a medical knowledgebase for similarity of the set of parameters with the one or more symptoms, wherein the medical knowledgebase comprises static medical resources, medical databases, online medical resources, and peer reviewed journals for a target disease; and,
predicting, by the AI engine (214), the disease based on the mapped one or more symptoms with the set of parameters associated with the target disease; and
facilitating, by the one or more processors (202), a final response corresponding to the predicted disease to the one or more users.

16. The method as claimed in claim 15, wherein the method further comprises the step of:
mapping, by the AI engine (214), said respective response with the information service; and transmitting, the information service in real-time to the plurality of first computing devices.

17. The method as claimed in claim 15, wherein the method further comprises the step of Modelling, by using the knowledge graph, one or more dependencies associated with one or more conditional queries to be received from the one or more users.

18. The method as claimed in claim 15, wherein the first set of attributes further comprises a dialogue state, an utterance state, a patient state, a disease state, and a symptom state, wherein the dialogue state comprises a conversation between the user (102) and the method (110), wherein the utterance state comprises one or more exchanges that occur between the user and the method, wherein the patient state comprises a complete information of the user, a plurality of responses to the set of input queries, wherein the disease state comprises a complete information about a target disease and wherein the symptom state comprises information about a plurality of symptoms associated with the target disease.

19. The method as claimed in claim 15, wherein the method further comprises the step of:
predicting, by the AI engine (214), the disease based on a scoring mechanism that comprises any or a combination of differential diagnosis of the Patient State, a symptom coverage score associated with a coverage of the one or more symptoms expressed by the user corresponding to the disease, a Relative Disease Score pertaining to relevance of the disease compared with other diseases and an Attribute Mismatch Decay score pertaining to a mismatch of the first set of attributes with the second set of attributes.

20. The method as claimed in claim 15, wherein the method further comprises the step of:
predicting, by the AI engine (214), the disease based on a plurality of contexts such as any or a combination of disease priors, risk factors, lab-tests, body organs, body methods.

21. The method as claimed in claim 15, wherein the method further comprises the step of:
providing, by the AI engine (214), an automatic learning of one or more weights and the set of parameters from the knowledge graph and a feedback analysis to self-improve prediction of the disease; and,
updating, by the AI engine (214), the knowledge graph based on a revised one or more weights in the knowledge graph as suggested by a plurality of medical experts.

22. The method as claimed in claim 15, wherein the method further comprises the step of
collecting, by the AI engine (214), a plurality of points of data chosen by a plurality of medical experts to gather, refine and enrich the medical knowledgebase.

23. The method as claimed in claim 18, wherein the method further comprises the step of:
obtaining, by the AI engine (214), any or a combination of a previous utterance and a response by the user for the previous utterance question for retrieval of information of a user from the dialogue state, wherein the previous utterance question is derived by the method from the received set of input queries;
obtaining, by the AI engine (214), one or more user answered symptoms from the symptom state;
obtaining, by the AI engine (214), a demography of the user from the patient state;
and obtaining, by the AI engine (214), from the disease state, a list of topmost diseases that relate with the one or more symptoms of the user with highest probability.

24. The method as claimed in claim 23, wherein the method further comprises the step of:
understanding, by the AI engine (214), the previous utterance to decide the next utterance based on any or a combination of a previous demographic utterance response, a previous symptom utterance response, and a previous attribute response.

25. The method as claimed in claim 15, wherein the method further comprises the step of:
filtering, by the AI engine (214), unrelated one or more symptoms from the knowledge graph;
determining, by the AI engine (214), one or more highly likely diseases based on the one or more symptoms of the user, the scores associated with the one or more symptom associated with the disease; and,
predicting, by the AI engine (214), an actual disease based on the filtering of unrelated one or more symptoms and determination the one or more highly likely diseases.
